(19) United States
(12) Patent Application Publication
(10) Pub. No.: US 2009/0197828 A1
(43) Pub. Date: Aug. 6, 2009

(54) SYSTEM, METHOD AND PACKAGE FOR PROVIDING A SUCROSE SOLUTION

(75) Inventor: PAUL C. DALY, ABINGTON, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/424,841

(22) Filed: Apr. 16, 2009

Related U.S. Application Data
(63) Continuation of application No. 09/670,781, filed on Sep. 27, 2000.

(51) Int. Cl. A61K 31/7016; A61P 25/00

(52) U.S. Cl. 514/53

(57) ABSTRACT

A solution of sucrose and water is packaged and placed in an aseptic state in a cup-shaped container with a removable cover for single patient use. A plurality of containers is shipped from a preparation site to a site of usage such as a hospital. A single container of the solution is opened at a site of a procedure for a neonatal infant, and the solution administered prior to the procedure as well as during or afterward, as needed for analgesic effect. Any residual solution is discarded after the procedure to prevent cross contamination of other patients.

### CROSS REFERENCE

This application is a continuation of U.S. application Ser. No. 09/670,781, titled “SYSTEM, METHOD and PACKAGE FOR PROVIDING A SUCROSE SOLUTION,” filed Sep. 27, 2000, the entire contents of which are hereby incorporated by reference.

### BACKGROUND OF THE INVENTION

1. Field of the Invention

The present invention relates to providing a sucrose solution having demonstrated analgesic and calming effects for use with neonatal infants and, more specifically, a system, method and package for providing such solutions in prepackaged, sterile form.

2. State of the Art

All newborn infants are subjected to a variety of medical procedures after birth. Such procedures include, by way of example only, vitamin K injections, immunization, circumcision, and venipuncture or heel stick for blood sampling. Preterm or ill infants experience additional, often painful and stressful, diagnostic procedures and treatments. However, only in rare instances do neonatal infants receive prophylactic analgesia.

Neonatal infants demonstrate a preference for sweet-tasting substances, including sucrose, fructose and glucose as well as artificial sweeteners. Intake of sucrose has demonstrated analgesic and calming effects on infants, and the other substances previously mentioned may have similar effects, but this has not been proven. On the other hand, lactose apparently does not induce analgesia or calming effects in newborn infants. Moreover, administration of oral sucrose has been proven to promote increased sucking and hand-to-mouth behavior in infants as well as reduce crying-related energy expenditure, the absence of which may positively affect feeding behavior and growth. No published studies of the analgesic or calming effect of dextrose are known to the inventor.

Current practice in hospitals employing substances such as sucrose, dextrose or even common table sugar is to mix up a large batch of solution in an on-site kitchen or pharmacy. As noted above, sucrose is the only sugar recognized uniformly to provide the desired analgesic and calming effects so, in some instances, administration of a sweet solution to infants is not efficacious. Moreover, the conditions in which these sweet solutions are mixed on site are by no means sterile, and the human traffic in the preparation environment increases an already substantial risk of contamination. Cross contamination between patients is also a problem, as doses of solution may be given to more than one patient from the same container.

Finally, even when sucrose is conventionally employed, formulations of the sweet solutions are not carefully controlled and, therefore, the desired results not always or even predictably achieved. Studies have indicated that the minimum concentration of sucrose needed to produce effective analgesia for procedural pain may be about 18%. Although such studies are not definitive, it has been established that too low a concentration of sucrose may not be efficacious. On the other hand, overly high dosages of sugars to infants are known to be detrimental.

It would thus be desirable to provide a technique for preparation and administration of sucrose solutions by clinicians in an effective manner and without the deficiencies attendant to conventional procedures.

### BRIEF SUMMARY OF THE INVENTION

The present invention comprises a system, method and package for providing sucrose solutions to neonatal infants.

According to the present invention, a solution of sucrose and water is formulated with a percentage of about 10% to about 50% sucrose, the remainder of the solution comprising water. The solution is metered into a cup or other container for single patient use or dosage. The product is packed aseptically or postprocess sterilized for safety and freshness, and leaves the preparation site in a sealed, sterile state. Multiple containers of packaged solution are boxed and shipped to the end user. At the site of usage, a container is opened and the solution administered prior to a painful or otherwise stressful procedure, for example, by dipping a pacifier in the opened container or drawing a small volume of solution into a dropper or syringe, the solution then being administered orally.

Other features and advantages of the present invention will become apparent to those of skill in the art through a consideration of the ensuing description, the accompanying drawings, and the appended claims.

### BRIEF DESCRIPTION OF THE SEVERAL VIEWS OF THE DRAWINGS

In the drawings, which illustrate what is currently considered to be the best mode for carrying out the invention:

FIG. 1 is a side sectional elevation of a cup-like container holding a volume of analgesic solution according to the present invention;

FIG. 2 is a partially cut away perspective view of a plurality of the cup-like containers of FIG. 1 holding solution; and

FIG. 3 is a flow diagram of a method of preparing and administering the analgesic solution according to the present invention.

### DETAILED DESCRIPTION OF THE INVENTION

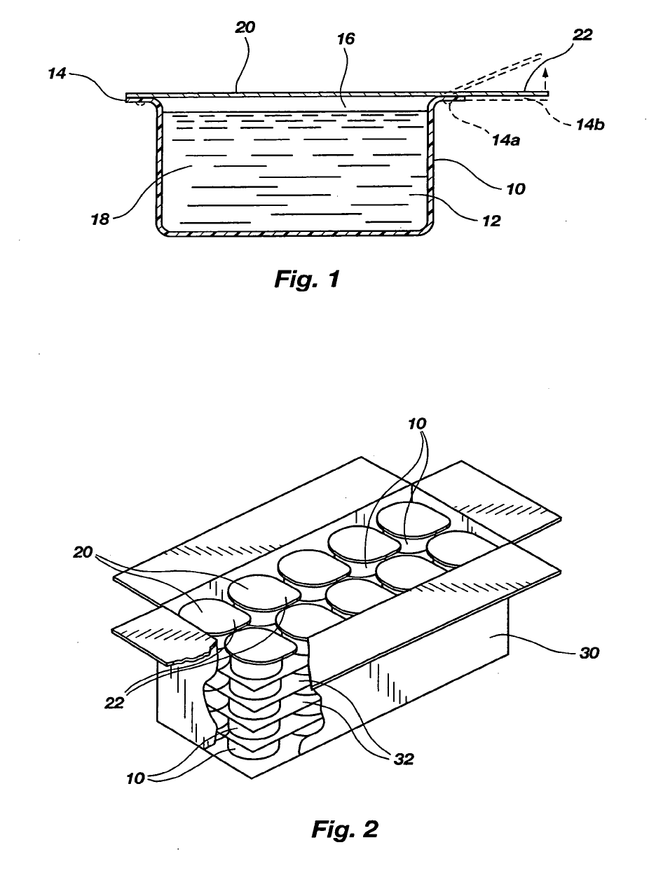

Referring now to FIG. 1 of the drawings, a thermally-formed or injection-molded polymer container 10 in a cup shape defining a cavity 12 and having a peripheral flange 14 at the mouth 16 thereof is filled, by way of example only, with about 40 ml of solution 18. After introduction of solution 18 into cavity 12, a cover 20 of a polymer film, a metal foil, a metallized insulating film or other suitable material is applied over mouth 16 and sealed to peripheral flange 14 by techniques known in the art. Peripheral flange 14 may have an annular indentation or groove in the top surface thereof as shown in broken lines 14a to facilitate cover 20 being sealed to peripheral flange 14 therealong by, for example, point contact with a heating tool. Cover 20 extends at least to an outer edge of peripheral flange 14. Container 10 as shown (see FIG. 2) is round, but other configurations such as square or rectangular with a like-shaped cover are contemplated. Suitable labeling (not shown) may be applied to the top of cover 20, as desired, for ease of viewing by the user.

Solution 18 may comprise a sucrose and water solution in the range of about 10% to about 50% sucrose, the remainder of the solution comprising water. The sucrose may be USP grade or clean sucrose, and the water clean or sterile. It is preferred currently by the inventor that solution 18 comprise about 24% USP grade liquid sucrose to about 76% clean water.

As noted above, the formulation and packaging of solution 18 may be performed aseptically or sterilization may be effected as a postprocess operation. Gamma irradiation is contemplated as one suitable postprocess sterilization technique. The manner of preparing and packaging analgesic solution 18 according to the invention is known to those of ordinary skill in the relevant art, so no further explanation thereof is deemed necessary.

FIG. 2 shows a plurality of sealed, cup-like containers 10 disposed in a box 30 for shipping. In the example shown, five groups of ten containers 10 each are layered in box 30 with spacer sheets 32 disposed between each layer, under the bottom layer and over the top layer. It may also be more easily seen from FIG. 2 that covers 20 of containers 10 include integral protrusions or tabs 22 extending substantially beyond the outer extent of peripheral flanges 14 at one side thereof, the remaining periphery of covers 20 substantially following the outer extent of flanges 14. If desired, flanges 14 may also include a tab or protrusion 14b of similar shape to protrusion or tab 22 as shown in broken lines in FIG. 1 to protect protrusion 22 from inadvertent lifting during handling and shipping. Protrusion 22 enables gripping by the user to facilitate peeling the cover 20 off of container 10 for access to solution 18 in cavity 12 through the wide mouth 16 as shown in broken lines. The relatively shallow depth and wide mouth configuration of container 10 is particularly advantageous for dipping of a pacifier end therein to coat it with solution 18 prior to insertion in an infant's mouth for the infant to suck. It may be desirable to configure container 10 as even wider and shallower than as currently depicted in the drawings to prevent tipping thereof if a pacifier is left therein between dosings. Similarly, the exemplary 40 ml volume of solution 18 in internal cavity 12 may be reduced to a lesser volume, for example 20 ml, as desired.

In accordance with the invention, it is preferred that a dose of no more than about 2 ml of solution 18 be administered to an infant for analgesia approximately two minutes prior to a planned procedure. If a pacifier is employed, it may be dipped in analgesic solution 18 and inserted in the infant's mouth. In such an instance, a dose of solution 18 may comprise about 0.2 ml. Recoating of the pacifier should only be effected as needed, not to exceed administration of the aforementioned 2 ml of solution 18. If administered by syringe or dropper, a few drops of solution 18 may be applied to the tongue or buccal surface. A dose volume of 0.05 to 2 ml is preferred. Repeat doses of solution 18, which may be administered during and immediately following the procedure, should not exceed the aforementioned 2 ml total volume. After the procedure, container 10 with residual solution 18 should be discarded to avoid any potential for cross contamination of other infants.

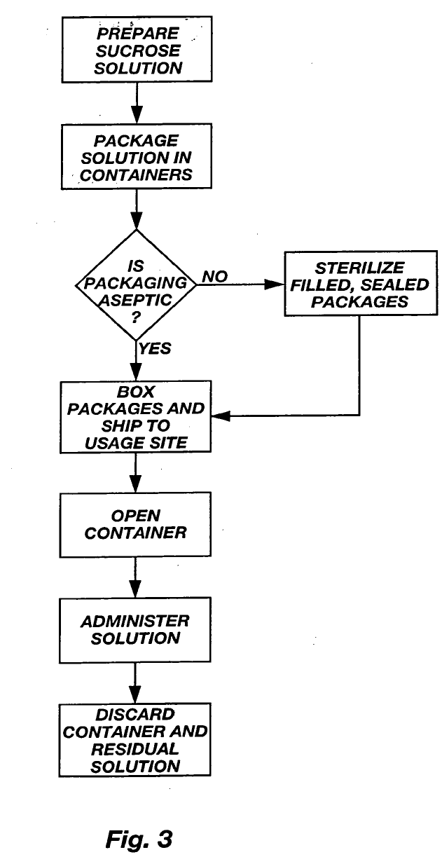

FIG. 3 comprises a flow diagram of an exemplary method of carrying out the present invention, comprising preparing the solution 18, packaging solution 18 in containers 10 either aseptically or with postprocess sterilization, boxing multiple containers 10 for shipment and shipping to a usage site (e.g., hospital), opening a container 10 in association with a planned procedure, administering the solution 18, and discarding any residual solution after the procedure. Of course, it would be possible to practice the invention by preparing solution 18 on site, packaging it aseptically and then using it on site. However, most if not all hospitals are not equipped to perform a packaging operation as contemplated by the invention.

While the present invention has been described with respect to an illustrated embodiment, those of ordinary skill in the art will understand and appreciate that additions and modifications to and deletions from the illustrated embodiment are possible without departing from the scope of the invention as encompassed by the claims herein.

What is claimed is:

1. A packaged solution for use in conjunction with a medical procedure on an infant, comprising:
a cup-shaped container defining a cavity therein opening to a mouth;
a volume of a solution within the cavity, the solution comprising water and about 10% to about 50% sucrose; and
a cover disposed over the mouth and sealing the solution within the cavity,
wherein the container has a container volume that is at least 50% larger than the volume of the solution, and
wherein the volume of the solution is larger than a volume of a unit dose of the solution for the medical procedure on the infant.

2. The packaged solution of claim 1, wherein:
the volume of the solution is 20 mL or larger;
the container volume is larger than 40 mL; and
the unit dose is 2 mL or less.

3. The packaged solution of claim 1, wherein the container volume is at least 100% larger than the volume of the solution.

4. The packaged solution of claim 3, wherein the volume of the solution is at least 300% larger than the volume of the unit dose.

5. The packaged solution of claim 1, wherein the unit dose comprises 0.05 to 2.0 mL of the solution.

6. The packaged solution of claim 5, wherein the volume of the solution is over 5 mL.

7. The packaged solution of claim 5, wherein the volume of the solution is over 10 mL.

8. The packaged solution of claim 1, wherein:
the container includes a peripheral flange about the mouth;
the cover is sealed to the peripheral flange; and
the cover includes a tab extending beyond the periphery of the flange such that a user can grasp and remove the cover.

9. The packaged solution of claim 1, wherein the solution and an interior of the container are in an aseptic state.

10. The packaged solution of claim 1, wherein the solution comprises about 24% USP grade liquid sucrose.

11. The packaged solution of claim 1, wherein the cup-shaped container has a greater width than depth.

12. The packaged solution of claim 1, wherein the mouth and cavity are sized to receive at least a portion of a pacifier therein.

13. A method for providing a solution in conjunction with a medical procedure on an infant, comprising:
opening an individual, single-use, sealed container having a volume of a solution therein, the solution comprising water and about 10% to about 50% sucrose, the container having a container volume that is at least 50% larger than the volume of the solution; and
administering a selected volume dose of the solution orally to the infant, wherein the volume of the solution is larger than a volume of the selected volume dose.

14. The method of claim 13, wherein the container volume is more than 100% larger than the volume of the solution, and wherein the volume of the solution is at least 100% larger than the selected volume dose.

15. The method of claim 13, further comprising, after administering the selected volume dose, discarding residual solution within the opened, individual, single-use container.

16. The method of claim 13, wherein administering the selected volume dose comprises dipping an object into the solution within the container and then administering the selected volume dose using the object.

17. The method of claim 16, wherein the object comprises a pacifier.

18. The method of claim 16, wherein opening the container comprises breaking a seal between a cover and a peripheral flange of the container by pulling on a tab of the cover that extends beyond the peripheral flange.

19. The method of claim 13, wherein the infant is a neonatal infant.

20. A packaged solution for use in conjunction with a medical procedure on an infant, comprising:
a cup-shaped container defining a cavity therein opening to a mouth that is sized to receive at least a portion of a pacifier therein, the container including a peripheral flange about the mouth, the container having a greater width than depth;
a volume of a solution within the cavity, the solution comprising water and between 10% and 50% sucrose; and
a cover disposed over the mouth and sealing the solution within the cavity, the cover being sealed to the peripheral flange, the cover including a tab extending beyond the peripheral flange such that a user can grasp and remove the cover,
wherein the container has a container volume that is at least 100% larger than the volume of the solution,
wherein the volume of the solution is over 10 mL,
wherein the volume of the solution is at least 400% larger than a volume of the unit dose of the solution for the medical procedure on the infant.

21. The packaged solution of claim 20, wherein the solution comprises about 24% USP grade liquid sucrose.
